(19) United States
(12) Patent Application Publication
(10) Pub. No.: US 2005/0089902 A1
(43) Pub. Date: Apr. 28, 2005

(54) Methods and compositions for siRNA expression

(75) Inventors: Lianxing Zheng, San Diego, CA (US); Sheng Ding, San Diego, CA (US); Peter G. Schultz, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Scripps Research Institute, La Jolla, CA (US)

(21) Appl. No.: 10/932,408

(22) Filed: Sep. 1, 2004

Related U.S. Application Data
(60) Provisional application No. 60/499,571, filed on Sep. 2, 2003.

(51) Int. Cl. C12Q 1/68; C12P 19/34

(52) U.S. Cl. 435/6; 435/91.2

(57) ABSTRACT

One-step methods for generating siRNA expression cassettes are provided. Expression cassettes useful for siRNA are also provided.

### CROSS-REFERENCE TO RELATED PATENT APPLICATIONS

The present patent application claims benefit of priority to U.S. Provisional Patent Application No. 60/499,571, filed Sep. 2, 2003, which is incorporated by reference for all purposes.

### BACKGROUND OF THE INVENTION

RNA interference (RNAi) is an evolutionarily conserved phenomenon in which gene expression is suppressed by the introduction of homologous double-stranded RNA (dsRNA). After dsRNA molecules are delivered to the cytoplasm of a cell, they are cleaved in vivo by the RNase III-like enzyme, Dicer, to 21-23 nucleotide (nt) small interfering RNAs (siRNAs). See, Bernstein, E., Caudy, A. A., Hammond, S. M. & Hannon, G. J. (2001) Nature 409, 363-6; Hammond, S. M., Bernstein, E., Beach, D. & Hannon, G. J. (2000) Nature 404, 293-6; Elbashir, S. M., Lendeckel, W. & Tuschl, T. (2001) Genes Dev 15, 188-200. These siRNAs are then incorporated into a multiple protein complex, the RNA induced silence complex (RISC). The duplex siRNA is unwinded, leaving the antisense RNA to guide the RISC to target the homologus mRNA, where the RISC associated endoribonuclease cleaves the target mRNA resulting in silencing of the target gene. See, Nykanen, A., Haley, B. & Zamore, P. D. (2001) Cell 107, 309-21; Martinez, J., Patkaniowska, A., Urlaub, H., Luhrmann, R. & Tuschl, T. (2002) Cell 110, 563-74. RNAi has been successfully used to suppress gene expression in a variety of organisms including zebrafish, C. elegans, Drosophila, planaria, mice and mammalian cells. See, Hannon, G. J. (2002) Nature 418, 244-51; Denli, A. M. & Hannon, G. J. (2003) Trends Biochem Sci 28, 196-201.

In C. elegans and Drosophila, RNAi is typically induced by the introduction of a long dsRNA (up to 1-2 kb) produced by in vitro transcription. This simple approach cannot be used in mammalian cells, where introduction of long dsRNA elicits a strong antiviral response obscuring any gene-specific silencing effect. Much of this response is due to activation of the dsRNA-dependent protein kinase PKR, which phosphorylates and inactivates the translation initiation factor eIF2a. However, introduction of 21 nt siRNAs with 3′ overhangs of 2 nts does not stimulate the anti-viral response in mammalian cells and can effectively target specific mRNAs for gene silencing. See, Elbashir, S. M., Harborth, J., Lendeckel, W., Yalcin, A., Weber, K. & Tuschl, T. (2001) Nature 411, 494-8.
siRNA molecules can be prepared by, for example, chemical synthesis or in vitro transcription. Alternatively, they can be transcribed in vivo using siRNA expression vectors or cassettes with a RNA polIII promoter (including U6, human H1, and tRNA promoters (Tuschl, T. (2002) Nat Biotechnol 20, 446-8; Brummelkamp, T. R., et al. (2002) Science 296, 550-3; Yu, J. Y., et al. (2002) Proc Natl Acad Sci USA 99, 6047-52; Sui, G., et al. (2002) Proc Natl Acad Sci USA 99, 5515-20; Paul, C. P., et al. (2002) Nat Biotechnol 20, 505-8; Lee, N. S., et al. (2002) Nat Biotechnol 20, 500-5; Kawasaki, H. & Taira, K. (2003) Nucleic Acids Res 31, 700-7; Miyagishi, M. & Taira, K. (2002) Nat Biotechnol 20, 497-500; Paddison, P. J., et al. (2002) Genes Dev 16, 948-58). Alternatively, a PolII promoter with a minimal poly A signal sequence can be used. See, Xia, H., et al. (2002) Nat Biotechnol 20, 1006-10. Tissue specific PolII promoters can be used to carry out tissue specific gene suppression.

Often, siRNA methods involve use of a single promoter is used to express a short hairpin sequence. In some cases, however, two polIII promoters are used to transcribe the sense and anti-sense siRNA sequences. However, previously-described expression systems suffer the disadvantage of not being able to construct siRNA expression library (randomized or mRNA-derived) easily in either pooled format or arrayed format. The present invention addresses this and other problems.

### BRIEF SUMMARY OF THE INVENTION

The present invention provides methods for producing two complementary strands of a tripartite DNA comprising a 5′ amplification primer binding site and a 3′ amplification primer binding site with an intervening sequence between the two sites. In some embodiments, the method comprises,
providing a polymerase extension reaction mixture comprising the following reagents:
   a) an initial double-stranded DNA [A/a] comprising
      a left end comprising a 5′ amplification primer binding site; and
      a right end complementary to a 3′ end of an intervening linking primer;
   b) the intervening linking primer [b] comprising:
      a 5′ end complementary to a left end of a terminal DNA;
      a 3′ end complementary to the right end of the initial DNA; and
      an intervening sequence between the 5′ end and the 3′ end;
   c) the terminal double-stranded DNA [C/c] comprising
      a right end that is a 3′ amplification primer binding site; and
      a left end complementary to the 5′ end of the intervening linking primer;
   d) DNA polymerase and components sufficient for a polymerase chain reaction;
reacting the reagents of the mixture in at least two thermocycles, wherein
   the first cycle comprises
      a melting temperature to denature double-stranded DNA;
      an initial annealing temperature to anneal the intervening linking primer [b] to a strand [A] of the initial double-stranded DNA; and
      an extension temperature for the polymerase to extend a 3′ end of the strand [A] of the initial double-stranded DNA using the intervening linking primer [b] as a template to produce an intermediate product [AB] comprising the strand [A] of the initial double stranded DNA and a complement [B] of the intervening linking primer; and
   the second cycle comprises
      a melting temperature to denature double-stranded DNA;
      an annealing temperature to anneal the intermediate product [AB] to a strand [c] of the terminal double-stranded DNA; and
      an extension temperature for the polymerase
         to extend the 3′ end of the intermediate product [AB] using the strand [c] of the terminal double-stranded DNA as a template to produce a single stranded tripartite DNA [ABC] comprising the strand [A] of the initial double stranded DNA, the complement [B] of the intervening linking primer, and the complement [C] of the strand of the terminal double stranded DNA sequences; and
         to extend the 3′ end of the strand [c] of the terminal double stranded DNA using the intermediate product [AB] as a template to produce the complement [abc] of the single-stranded tripartite DNA. For convenience, the bracketed letters refer to DNA strands depicted in FIG. 6.

In some embodiments, the polymerase extension reaction mixture further comprises
a) a 5′ amplification primer that anneals to the 5′ amplification primer binding site; and
b) a 3′ amplification primer that anneals to the 3′ amplification primer binding site; and
and the method further comprises at least one additional thermocycle following the second thermocycle, wherein the additional thermocycle comprises a differential annealing temperature at which the 5′ amplification primer anneals to the 5′ amplification primer binding site and the 3′ amplification primer anneals to the 3′ amplification primer binding site, but the intervening linking primer does not anneal to the strand of the initial double-stranded DNA, thereby amplifying the two complementary strands of a tripartite DNA.

In some embodiments, the initial DNA comprises a first pol III promoter and pol III terminator and the terminal DNA comprises a second pol III promoter and pol III terminator; and the method links the initial and terminal DNAs such that the promoters are oriented towards each other. In some embodiments, the first pol III promoter is the U6 promoter and the second pol III promoter is the H1 promoter. In some embodiments, the method further comprises cloning the linked DNA sequences into a vector.

In some embodiments, the 5′ and 3′ amplification primers comprise restriction enzyme recognition sequences. In some embodiments, the method comprises cleaving the tripartite DNA sequences with a restriction enzyme that cleaves at least one restriction enzyme recognition sequence and ligating the cleaved DNA sequence into a vector.

In some embodiments, the mixture comprises a plurality of different intervening linking primers comprising different intervening sequences. In some embodiments, the different intervening linking primers comprise randomly-generated intervening sequences.

In some embodiments, the initial DNA comprises a first pol III promoter and pol III terminator and the terminal DNA comprises a second pol III promoter and pol III terminator; the method links the polynucleotide sequences such that the promoters are oriented towards each other; and the resulting tripartite DNAs are cloned into a vector, thereby synthesizing a library of random intervening sequences between the first and second pol III promoters.

In some embodiments, the method further comprises introducing the amplified sequences into cells under conditions resulting in expression from the pol III promoters in the cell, thereby forming double-stranded RNA.

In some embodiments, the intervening linking primer is between 40-70 nucleotides long. In some embodiments, the intervening linking primer is between 61-63 nucleotides long.

In some embodiments, the initial annealing temperature is about 55° C. In some embodiments, the differential annealing temperature is about 63° C.

In some embodiments, the initial DNA and the terminal DNA are part of one polynucleotide. In some embodiments, the initial DNA and the terminal DNA are part of different polynucleotides.

The present invention also provides kits for generating a siRNA polynucleotide. In some embodiments, the kit comprises,
a) an initial double-stranded DNA comprising
   a left end comprising a 5′ amplification primer binding site; and
   a right end complementary to a 3′ end of an intervening linking primer;
b) the intervening linking primer comprising:
   a 5′ end complementary to a left end of a terminal DNA; and
   a 3′ end complementary to the right end of the initial DNA;
   an intervening sequence between the 5′ end and the 3′ end;
c) the terminal double-stranded DNA comprising
   a right end that is a 3′ amplification primer binding site; and
   a left end complementary to the 5′ end of the intervening linking primer;
d) a 5′ amplification primer; and
e) a 3′ amplification primer.

In some embodiments, the kit further comprises a DNA polymerase. In some embodiments, the 5′ and 3′ amplification primers comprise restriction enzyme recognition sequences.

In some embodiments, the kit comprises a plurality of different intervening linking primers comprising different intervening sequences. In some embodiments, the different intervening linking primers comprise randomly-generated intervening sequences.

In some embodiments, the first pol III promoter is the U6 promoter and the second pol III promoter is the H1 promoter. In some embodiments, the intervening linking primer is between 40-70 nucleotides long. In some embodiments, the intervening linking primer is between 61-63 nucleotides long.

In some embodiments, the initial DNA and the terminal DNA are part of one polynucleotide. In some embodiments, the initial DNA and the terminal DNA are part of different polynucleotides.

The present invention also provides mixtures comprising,
a) an initial double-stranded DNA comprising
   a left end comprising a 5′ amplification primer binding site; and
   a right end complementary to a 3′ end of an intervening linking primer;
b) the intervening linking primer comprising:
   a 5′ end complementary to a left end of a terminal DNA; and
   a 3′ end complementary to the right end of the initial DNA;
   an intervening sequence between the 5′ end and the 3′ end;
c) the terminal double-stranded DNA comprising
   a right end that is a 3′ amplification primer binding site; and
   a left end complementary to the 5′ end of the intervening linking primer;
d) a 5′ amplification primer; and
e) a 3′ amplification primer.

In some embodiments, the kit further comprises a DNA polymerase. In some embodiments, the 5′ and 3′ amplification primers comprise restriction enzyme recognition sequences.

In some embodiments, the mixture comprises a plurality of different intervening linking primers comprising different intervening sequences. In some embodiments, the different intervening linking primers comprise randomly-generated intervening sequences. In some embodiments, the first pol III promoter is the U6 promoter and the second pol III promoter is the H1 promoter.

In some embodiments, the intervening linking primer is between 40-70 nucleotides long. In some embodiments, the intervening linking primer is between 61-63 nucleotides long.

In some embodiments, the initial and terminal DNAs are part of one polynucleotide. In some embodiments, the initial and terminal DNAs are part of different polynucleotides.

### Definitions

The term “siRNAs” refers to short interfering RNAs. In some embodiments, siRNAs comprise a duplex, or double-stranded region, where each strand of the double stranded region is about 18 to about 25 nucleotides long; the double stranded region can be as short as, e.g., 16, and for mammalian systems are typically no longer than about 29, base pairs long, where the length is determined by the antisense strand. However, as demonstrated herein, siRNAs can be 100s of base pairs long in some systems. Often siRNAs contain from about two to four unpaired nucleotides at the 3′ end of each strand. siRNAs appear to function as key intermediaries in triggering RNA interference in invertebrates and in vertebrates, and in triggering sequence-specific RNA degradation during posttranscriptional gene silencing in plants. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. One strand of the double stranded region need not be the exact length of the opposite strand; thus, one strand may have at least one fewer nucleotides than the opposite complementary strand, resulting in a “bubble” or at least one unmatched base in the opposite strand. One strand of the double stranded region need not be exactly complementary to the opposite strand; thus, the strand, preferably the sense strand, may have at least one mismatched base-pair.

The term “annealing temperature” is used as it is commonly used by practitioners of the polymerase chain reaction, and refers to a temperature at which a given polynucleotide hybridizes to a complementary nucleotide sequence in a given environment, e.g., a buffer used for amplification reactions.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids.

An “amplification reaction” refers to any chemical, including enzymatic, reaction that results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991), rolling circle amplification (RCA) (Lisby, Mol. Biotechnol. 12(1):75-99 (1999)); Hatch et al., Genet. Anal. 15(2):35-40 (1999)) and branched DNA signal amplification (bDNA) (see, e.g., Iqbal et al., Mol. Cell Probes 13(4):315-320 (1999)).

The phrase “nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“Annealing primers” refers to providing sufficient conditions (e.g., a low enough temperature in an amplification reaction) that allows for primers to hybridize to a complementary sequence on a template polynucleotide, e.g., in a PCR reaction. Primer annealing temperatures are commonly between 50 and 65° C.

“Activating a polymerase” or “extending a DNA polymerase” in the presence of a polymerase, as used herein, refers to providing sufficient conditions (e.g., a temperature in an amplification reaction) that allows for polymerase processivity. Generally, an optimal temperature for Taq polymerase activation in PCR reactions is 72° C.

“Denaturing DNA strands” refers to providing sufficient conditions (e.g., a sufficiently high temperature in an amplification reaction) that allows for dissociation of complementary DNA strands. A typical dissociation temperature in PCR reactions is 94° C.

A “thermal cycle” or “thermocycle” refers to a set of temperature changes that repeat back to a starting temperature. For example, a typical PCR thermocycle is a dissociation (e.g., ˜94° C.) temperature, an annealing temperature (e.g., ˜63 or ˜55° C.) and a polymerase activation temperature (e.g., ˜72° C.). The cycle can then be repeated for a determined number of rounds.

“Linking” two polynucleotide sequences as used herein refers to covalently attaching the two polynucleotide sequences by introducing a third polynucleotide sequence between the sequences, thereby forming one nucleic acid that comprises all three sequences. In these embodiments, the third polynucleotide sequence between the other two sequences is referred to an “intervening linking primer.”

A “pol III promoter” refers to a promoter that is recognized by RNA polymerase III to initiate transcription. Examples of pol III promoters include, e.g., human and mouse H1 promoters, the human U6, 5S, U6, adenovirus VA1, Vault, telomerase RNA, and tRNA gene promoters. In addition, several other pol III promoter elements have been reported including those responsible for the expression of Epstein-Barr-virus-encoded RNAs (EBER), and human 7SL RNA. “Pol III terminators” refer to sequences recognized by RNA polymerase III to terminate transcription. Exemplary pol III terminators include four or five contiguous thymidines (e.g., TTTT or TTTTT).

A “primer binding site” refers to a sequence comprising sufficient nucleotides complementary to a primer to allow for annealing in a PCR reaction. The binding site in a double stranded DNA can be either strand.

The terms “right” and “left” as used to refer to the ends of DNAs are used solely to differentiate the two ends of the DNAs.

### BRIEF DESCRIPTION OF THE DRAWINGS

FIG. 1A illustrates a strategy for generating siRNA using two opposing Pol III promoters. To construct siRNA vector (referred to as “pDual”), the mouse U6 and human H1 promoter sequences were cloned into pBluescript SK in opposite directions. Appropriate mutations were made to define termination signals for siRNA transcription or facilitate inserting siRNA encoding sequences. To create a gene specific siRNA expression plasmid, a pair of complementary oligonucleotides (35 to 37 nt) were annealed and ligated into pDual digested with Bgl II and Hind III.

FIG. 1B illustrates a single step PCR strategy for producing siRNA expression cassettes based on the pDual system. An oligonucleotide primer (referred to herein as the “third” primer) encoding the desired siRNA sequence was used to bridge the U6 and H1 promoter fragments (templates). In addition, two 31mer universal primers (common to all PCR reactions) complementary to the 5′ ends of the U6 and H1 promoters were also added to the PCR reaction. Note that the complete third primer sequence is not shown. Only the siRNA portion and a few complementary nucleotides are depicted.

FIG. 2 illustrates the specific and efficient suppression of transfected firefly luciferase by siRNA expressed using the pDual system in 293T cells. Number “5” indicates empty pDual used as control. Numbers “1,” “2,” and “3” represent siRNAs expressed by pDual. siRNA sequences of 19 nt in length that correspond to three different regions of the firefly luciferase gene were chosen as the target sequences and cloned into pDual. Number “4” represents hairpin siRNA expressed by the vector pSuper. For comparison, siRNA coding sequences in (1) and (4) are the same.

FIGS. 3A and 3B illustrate specific and efficient suppression of firefly luciferase expression by PCR-derived siRNA expression cassettes based on the pDual system. Comparison of gene suppression effects by siRNAs derived from pDual vector and PCR fragments in HEK-293T cells (FIG. 3A) or P19 cells (FIG. 3B). Cells were transfected with pGL3/pRL-SV40 and pDual-Luc (2 in A&B), or pGL3/pRL-SV40 and PCR-derived siRNA expression cassette (4 in A&B). pDual and PCR fragment derived from pDual are used as control (1 and 3 in A&B).

FIGS. 4A and 4B illustrate specific and efficient suppression of transfected firefly luciferase by long double-stranded siRNA expressed in P19 cells using either the pDual vector system or PCR-derived siRNA expression cassettes based on the pDual system. FIG. 4A illustrates the comparison of gene suppression by double-stranded interfering RNAs of different length expressed by pDual. FIG. 4B illustrates comparison of gene suppression by double-stranded interfering RNAs of different length expressed by PCR fragments derived from pDual.

FIG. 5 depicts a method of producing a random siRNA expression library. A library of third primers with a randomized siRNA sequence was chemically synthesized. This set of primers and another primer complementary to the 5′ end of H1 promoters were used to amplified the H1 promoter. The PCR product was then enzyme digested and ligated into a vector that already has U6 promoter in it. This random siRNA expression library can then be used to do genetic screening as described herein.

FIG. 6 illustrates the mechanism of producing a tripartite DNA comprising two DNAs (an initial (A/a) and a terminal (C/c) DNA) linked by an intervening sequence provided by an intervening linking primer (b). Capital letters represent one strand whereas the same small case letter indicates the complementary strand.

### DETAILED DESCRIPTION OF THE INVENTION

I. Introduction

The present invention provides a novel dual promoter siRNA expression system which allows for the facile construction of siRNA expression libraries for genome-wide screens. In some embodiments, a gene specific siRNA sequence is inserted between two different opposing promoters. As discussed in the examples, the promoters can each be Pol III promoters such as the mouse U6 and human H1 promoters. Upon transfection into mammalian cells, the sense and antisense strands of the siRNA duplex are transcribed by these two opposing promoters from the same template, resulting in a siRNA duplex, which in some embodiments has a two uridine overhang on each 3′ terminus, similar to the siRNA generated by Dicer. These siRNAs can be incorporated into the RISC without any further modification. The siRNAs transcribed by this vector can induce strong and specific gene suppression of both endogenous genes and ectopically expressed genes. Furthermore, a single step PCR protocol is described which allows for the production of siRNA expression cassettes in a high throughput manner. These PCR-derived, non-hairpin-based siRNA expression cassettes have also been shown to induce specific and strong suppression of endogenous and ectopically expressed gene function when transfected into mammalian cells.

II. Methods of Linking Two DNA Sequences

The present invention provides methods of producing and amplifying a tripartite DNA product comprising two polynucleotides, arbitrarily designated an “initial DNA” and a “terminal DNA,” and an intervening sequence, using three primers in a thermocyclic amplification reaction (e.g., the polymerase chain reaction). The initial and terminal DNAs are generally provided as double stranded molecules, but can also be provided as single-stranded nucleic acids. The termini of the DNAs are referred to as “right” and “left” to differentiate the two ends. Two of the primers are referred to as 5′ and 3′ amplification primers and bind to primer binding sites on the initial and terminal DNAs, respectively. All of the primers are generally provided in molar excess for amplification reactions.

The third primer is referred to as an “intervening linking primer,” which has a 5′ end complementary to the left end of the terminal DNA, and a 3′ end that is complementary to the right end of the initial DNA. The intervening linking primer also comprises an intervening sequence between its 5′ and 3′ ends. The intervening sequence can be any desired sequence. As discussed in more detail below, in some embodiments, the intervening sequence is an siRNA sequence, typically between, e.g., 16-29 and often 18-25 or 21-23 nucleotides long. In other embodiments, the intervening sequence can be any size, including 100 nucleotides or more. In such cases, while referred to as a primer for the purposes of this application, those of skill will understand that such long sequences are best synthesized as a double-stranded polynucleotide using molecular biological methods (e.g., PCR) rather than by chemical synthesis.

The 5′ and 3′ end sequences of the third primer are designed to have a higher annealing temperature for hybridizing to the ends of the initial and terminal DNAs, than the 5′ and 3‘amplification primers’ annealing temperature for hybridizing to the initial and terminal DNAs. In some embodiments, the 5′ and 3′ end sequences of the intervening linking primer will each have at least 7, 8, 9, 10, 11, 12, 13, 14, 15, 16, 17, 18, 19, 20, 21, 22, 23, 23, 25 or more nucleotides that hybridize to the polynucleotide sequence termini. In some cases, the 5′ and 3′ ends will have no more than about 30 and sometimes no more than about 22, 20, or 18 nucleotides complementary to the polynucleotide sequence termini. Thus, in some cases, the entire length of the third primer is about 40-70 nucleotides and sometimes between 61-63 nucleotides.

In brief, the initial and terminal DNAs and three primers are provided in a mixture comprising components sufficient for themocyclic amplification. The mixture of amplification components, initial and terminal DNAs, and three primers are submitted to thermocyclic conditions sufficient for amplification. The thermocyclic conditions comprise at least two thermocycles.

The first thermocycle produces an intermediate product and comprises
a melting temperature to denature double-stranded DNA;
an initial annealing temperature to anneal the intervening linking primer to a strand of the initial double-stranded DNA; and
an extension temperature for the polymerase to extend a 3′ end of the strand of the initial double-stranded DNA using the intervening linking primer as a template to produce an intermediate product comprising the strand of the initial double stranded DNA and a complement of the intervening linking primer.

The second cycle uses the intermediate product to form a tripartite DNA product and comprises
a melting temperature to denature double-stranded DNA;
an annealing temperature to anneal the intermediate product to a strand of the terminal double-stranded DNA; and
an extension temperature for the polymerase
   to extend the 3′ end of the intermediate product using the strand of the terminal double-stranded DNA as a template to produce a single stranded tripartite DNA comprising the strand of the initial double stranded DNA, the complement of the intervening linking primer, and the complement of the strand of the terminal double stranded DNA sequences; and
to extend the 3′ end of the strand of the terminal double stranded DNA using the intermediate product as a template to produce the complement of the single-stranded tripartite DNA.

The annealing temperatures in the first two cycles is sufficiently low (e.g., 55° C.) to allow hybridization of the intervening linking primer to the initial and terminal DNAs. Ideally, a sufficient number of cycles at the first annealing temperature are performed to allow for sufficient accumulation of the tripartite DMA product to act as a template. However, accumulation of too much of the intermediate is not desirable because significant quantities will contaminate the final amplification product. In some embodiments, 1, 2, 3, 4, 5, 6, 7, 8, 9, 10, or more cycles are performed at the first annealing temperature.

Subsequently, the mixture is submitted to at least one thermocycle in which the annealing temperature is higher (e.g., 63° C.) than the annealing temperature of the intervening linking primer to the initial or terminal DNAs. In this portion of the amplification reaction, only the 5′ and 3′ amplification primers prime the amplification reaction, thereby amplifying the tripartite product. A sufficient number (e.g., 10-50, usually 20-40) of cycles are performed at this second annealing temperature to allow for accumulation of the final amplification product, i.e. two polynucleotide sequences linked by the intervening sequence of the third primer.

Optionally, the 5′ and 3′ amplification primers will comprise unique restriction endonuclease recognition sequences at their 5′ end to allow for efficient cloning of the amplification products. For example, following amplification, the amplification product can be cleaved with the restriction enzymes and then cloned into a vector, e.g., plasmid, retroviral vector, adenoviral vector, etc.

In some embodiments, the initial and terminal DNAs are part of separate nucleic acids. Alternatively, the two DNAs represent two sequences of one nucleic acid, e.g., different parts of a plasmid that has been cleaved at a cloning site.

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)). Polynucleotide amplification methods are well known. See, e.g., PCR Protocols: A Guide to Methods and Applications (Innis et al., eds, 1990).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

III. Generation of siRNAs

The present invention is particularly useful for generating siRNAs. For example, the methods of the invention can be used to generate siRNAs expression cassettes comprising two opposing promoters positioned to express complementary strands of an intervening sequence provided by the intervening linking primer as discussed above. In preferred embodiments, the two promoters are different, thereby reducing the likelihood that the two promoters will hybridize to each other.

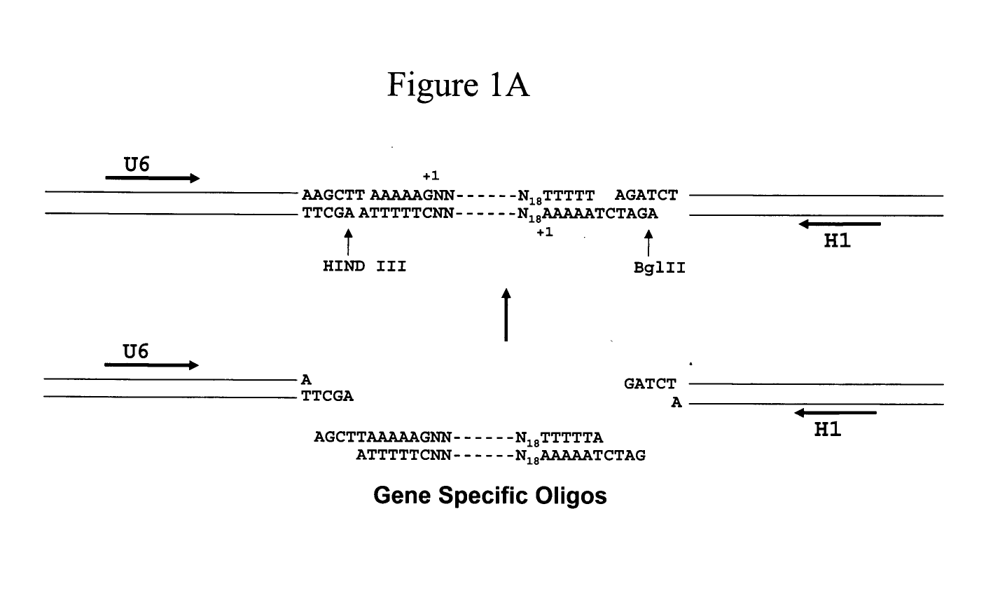

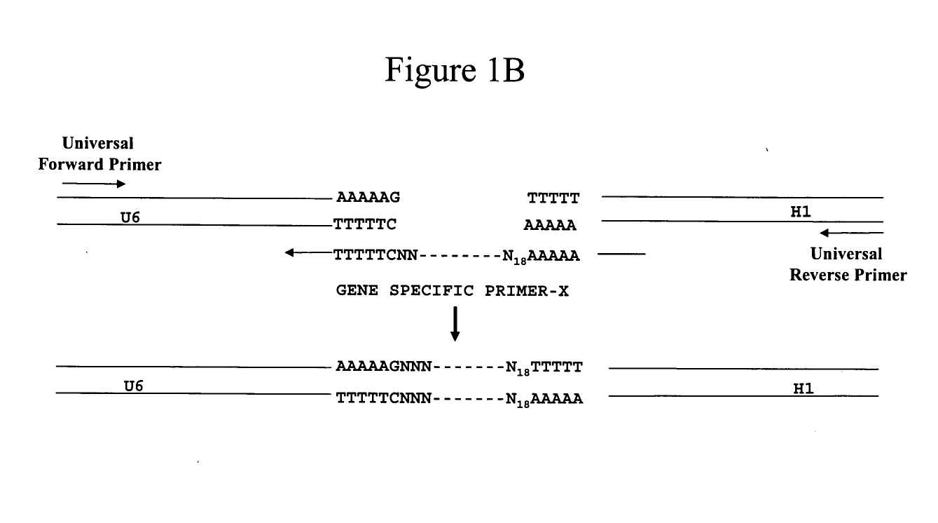

In some embodiments, the promoters are pol III promoters. One advantage of pol III promoters is that pol III terminators are rather short, allowing for insertion of terminators flanking the DNA between the promoters. See, e.g., FIGS. 1A and 1B. In the embodiments illustrated in FIGS. 1A and 1B, pol III promoters H1 and U6 are provided. In the examples, the initial DNA comprises the U6 promoter and the terminal DNA comprises the H1 promoter. Those of skill will understand that any pol III promoters can be used in this configuration. Exemplary pol III promoters include the human and mouse H1 promoters the human U6 promoter and the 5S, U6, adenovirus VA1, Vault, telomerase RNA, and tRNA gene promoters. Several other pol III promoter elements have been reported including those responsible for the expression of Epstein-Barr-virus-encoded RNAs (EBER), and human 7SL RNA. Any of these RNA polymerase III promoters, or functional derivatives thereof, may be used in the present invention to drive expression of the siRNA.

In addition, the RNA polymerase III promoter constructs of the invention may comprise various elements to allow for tissue specific, or temporally (time) specific expression. Methods to achieve such tissue or temporally controlled expression are known in the art and any of these may be employed to achieve such expression. By using such mechanisms this may allow the inhibition of the target gene to occur in a specific cell type or stage of development. This can have applications in both therapy and developmental biology for example, where the aberrant expression or mutated allele is only being expressed in a particular cell type or if it is not wished to disrupt expression in other cell types or where a gene is only expressed during a particular stage of embryonic development or maturation of the adult organism. Such promoters also allow for the study of essential embryonic genes in mature adults.

The methods of the invention using the promoters described above provide a one-step PCR method for generating siRNA expression cassettes. In these methods, an initial DNA comprises a first promoter and, optionally, a terminator corresponding to a second promoter, and a terminal DNA comprises a second promoter and, optionally, a terminator corresponding to the first promoter are provided in an amplification mixture. A primer complementary to each promoter is provided in the mixture. An intervening linking primer is also provided comprising a 5′ end complementary to the terminal DNA and a 3′ end complementary to the initial DNA and an intervening sequence comprising a target siRNA sequence. The target siRNA sequence is typically between, e.g., 16-29, 18-25 or 21-23 nucleotides long and in some embodiments is at least partially complementary to an mRNA sequence that is to be silenced. Of course, random sequences can also be used to identify the genes involved in producing a phenotype. Performing the thermocycling reaction with two different annealing temperatures, as described above, results in an siRNA expression cassette with two opposing promoters separated by an intervening siRNA sequence and, optionally, terminators. Pol III terminators are known in the art and can include, e.g., a series (e.g., 4 or 5 or more) of T's. See, e.g., FIG. 1B.

IV. Uses of siRNAs

The present invention provides methods of transfecting a mammalian cell with an expression cassette or with a vector as described above. The present invention also provides methods of expressing siRNA in a mammalian cell by transfecting the cell with an expression cassette or with a vector. The present invention also provides methods of silencing a gene in a mammalian cell by transfecting the cell with an expression cassette or with a vector as described above, where the siRNA encoded by the expression cassette targets a gene. In these methods, the cell is transfected transiently or stably. Moreover, in these methods, the target of the siRNA may be an endogenous gene, an exogenous gene, such as a viral or pathogenic gene or a transfected gene, or a gene of unknown function.

The target gene may be any gene of which it is desired to inhibit or modulate the function of. The purpose of the inhibition may be therapeutic, prophylactic, or to study the function of a particular gene. The inhibition of the gene may be to alter the phenotype of a cell or organism in some desired way. Typically, the target gene will be a eukaryotic gene, but alternatively the target gene may be prokaryotic such as a viral gene being expressed in a eukaryotic host cell. The target gene may encode a polypeptide or alternatively a structural or enzymatic RNA. However, preferably the target gene encodes a polypeptide.

The target gene can be, e.g., a developmentally important gene, or encode a cytokine, lymphokine, a growth or differentiation factor, a neurotransmitter, an oncogene, a tumor suppressor gene, a membrane channel, or component thereof. The gene can encode a receptor and in particular one for the gene products of any of the genes mentioned herein. The target gene can be one involved in apoptosis. Those of skill in the art will recognize that the above list is not exhaustive. Typically, the target gene will be one associated with a disease or disorder and the methods of the invention may be used to treat, prevent, or ameliorate that disease or disorder.

In many embodiments of the invention it will be desired to check the efficacy of the siRNAs in blocking the expression of the target gene. The inhibition of the gene may be measured in a variety ways, typically at the RNA, protein or phenotypic level.

Inhibition may be confirmed using biochemical techniques such as Northern blotting, nuclease protection, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

Inhibition in a cell line or whole organism, may be measured by using a reporter or drug resistance gene whose protein product is easily assayed. Such reporter genes and selection markers include any of those mentioned herein. Inhibition may also be measured at the phenotypic level. For example, the appearance of a phenotype similar to that associated with disruption of the targeted gene may be looked for. Where the purpose of the siRNA is to block expression of a gene associated with a disease, whether or not the disease is prevented, ameliorated or treatable using the siRNA, may be measured. Where the purpose of the siRNA is to treat an infectious disease, any reduction in viral or bacterial load may be assessed or alternatively the presence, absence or severity of symptoms associated with the disorder may be measured.

The present invention also provides methods of transfecting a mammalian cell with an expression cassette or with a vector as described above. The present invention also provides methods of expressing siRNA in a mammalian cell by transfecting the cell with an expression cassette or with a vector as described above. The present invention also provides methods of silencing a gene in a mammalian cell by transfecting the cell with an expression cassette or with a vector as described above, where the siRNA encoded by the expression cassette targets a gene. In these methods, the cell is transfected transiently or stably, and the cell is a cultured mammalian cell, and in some embodiments, a human cell.

The compositions and methods of the present invention are applicable to the field of reverse genetic analysis, e.g., by gene silencing. An siRNA construct can be designed to silence a gene of unknown function, inserted into an expression cassette, and transfected into the cell in which the target gene is expressed. The effect of the lack of or disappearance of an expressed gene product in the transfected cell can then be assessed; such results often lead to elucidation of the function of the gene. Application of siRNA to genes of known function is also contemplated to further examine the effects of the absence of the targeted gene function in a transfected cell.

V. Generation of siRNA libraries

The expression cassettes and vectors of the invention can also be used to generate large collections of siRNAs to perform genome-wide screens for genes that act in biologically relevant pathways. For example, libraries of siRNAs can be generated in a single-step PCR reaction using a plurality of different intervening linking primers, e.g., with common ends for hybridization to the two promoter polynucleotides and different intervening sequences, using the methods described herein, e.g., in section III. Genetic “loss-of-function phenotype” screens using such libraries may yield novel therapeutic targets that are candidates for drug development or may be used to evaluate the contribution of a limited number of candidate genes to a biological response.

Phenotypic genetic screens using cDNA expression libraries have been very successful for selection of genes that act in a dominant fashion to modulate cell behavior. The siRNA gene libraries allow, for the first time, a genome-wide evaluation for loss-of-function phenotypes in mammalian systems. This means that the equivalent of a homozygote for a recessive mutation may be generated.

Sequences to be inserted in the siRNA vector of the invention can be selected in silico by screening the appropriate databases for unique short nucleotide sequences, of the lengths specified herein for the double stranded region of the siRNA of the invention, such as 19mers, 20mers, 21mers, etc., for every known gene and every EST or a substantial proportion of these.

Such libraries may be based on human gene sequences for use in human cell systems or of species such any of those mentioned herein and in particular those of mammalian origin, or alternatively pathogenic origin such as viral origin.

Libraries of siRNAs can be introduced into the appropriate cell system and a response of the cells can be monitored. Any of the assays mentioned herein may be used to monitor the cells. The cells that show an altered response can be identified in various ways, depending on the nature of the biological system, and the siRNA that is expressed in the identified cell type can be recovered by several strategies, including PCR-based amplification of the specific siRNA insert using vector-specific primers.

VI. Kits and Mixtures

This invention also provides kits for the amplification and/or use of siRNA expression cassettes. The kits can include, e.g., a container and
a) an initial double-stranded DNA comprising
   a left end comprising a 5′ amplification primer binding site; and
   a right end complementary to a 3′ end of an intervening linking primer;
b) the intervening linking primer comprising:
   a 5′ end complementary to a left end of a terminal DNA; and
   a 3′ end complementary to the right end of the initial DNA;
   an intervening sequence between the 5′ end and the 3′ end;
c) the terminal double-stranded DNA comprising
   a right end that is a 3′ amplification primer binding site; and
   a left end complementary to the 5′ end of the intervening linking primer;
d) a 5′ amplification primer; and
e) a 3′ amplification primer.

In some cases, the kits will also comprise components sufficient for thermocyclic amplification such as a DNA polymerase (e.g., Taq, nucleotides, buffers, etc.). In some cases, the first and second primers comprise restriction enzyme recognition sequences. In some embodiments, the mixture comprises a plurality of different primers each comprising a 5′ end and a 3′ end, wherein the 5′ end is complementary to the first terminus and the 3′ end is complementary to the second terminus. In some embodiments, the different primers comprise randomly-generated sequences between the 5′ and 3′ ends.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

Design of Dual promoter vector. The design of the siRNA expression vector (referred to as “pDual”) features two opposing RNA pol III promoters to direct the transcription of the sense and antisense siRNA sequences from an interposed gene-specific sequence. To avoid vector instability which might be caused by two complementary promoter sequences flanking the siRNA encoding sequence, two different pol III promoters, the mouse U6 and human H1 promoters, were chosen. Five Ts were inserted at the 3′ end of the two opposing promoters as a termination signal since it is known that for the H1 promoter the last five nucleotides near the transcriptional start site can be substituted by unrelated sequences without affecting promoter activity. See, e.g., Hannon, G. J., et al. (1991) J Biol Chem 266, 22796-9; Baer, M., Nilsen, et al. (1990) Nucleic Acids Res 18, 97-103. To construct the pDual vector, the mouse U6 and human H1 promoter sequences were cloned into pBluescript SK in opposite direction (FIG. 1A). To facilitate the cloning of siRNA encoding sequences into the vector, Hind III and Bgl II sites were created in the promoter sequences flanking the insertion site by site-directed mutagenesis. All mutations created in the promoter sequences, including the -TTTTT-substitution, did not affect the position of the transcriptional start site relative to the TATA box.

To create a gene-specific siRNA expression plasmid, a pair of complementary oligonucleotides (35 to 37 nt) were annealed and ligated into pDual digested with Bgl II and Hind III. In the resulting plasmid, 19 bp to 21 bp of the siRNA encoding sequence is flanked by five As at the 5′ end and five Ts at the 3′ end, and the five consecutive Ts function as a termination signal for the pol III promoters. Once transfected into mammalian cells, the sense and antisense strands are transcribed by two opposing promoters on the same template, resulting in 19 to 21 base pair RNA duplex with a TT overhang at the 3′ end, closely resembling the Dicer digested product.

PCR protocol for high throughput production of siRNA expression cassettes. In order to produce the siRNA expression cassettes, we developed a single step PCR protocol based on our dual promoter vector (FIG. 1B). An oligonucleotide encoding the desired siRNA sequence was used to bridge the U6 and H1 promoters fragments. In addition, two 31mer universal primers complementary to the 5′ ends of the U6 and H1 promoters respectively, were also added to the PCR reaction. The later two primers are common to all PCR reactions. In order to efficiently produce the desired PCR product, a PCR protocol was used that involves two separate cycling steps with different annealing temperatures (see Methods). In the first five cycles, low temperature annealing at 55° C. allows the three primers to anneal with the corresponding templates. In the following 36 cycles, the annealing temperature is raised to 63° C. such that only the two universal primers are able to anneal with their templates, resulting in amplification of only the full length siRNA expression cassette. Under these conditions, the major PCR product is the 650 bp full length siRNA expression cassette (>95% purity judged by agarose gel and sequencing).

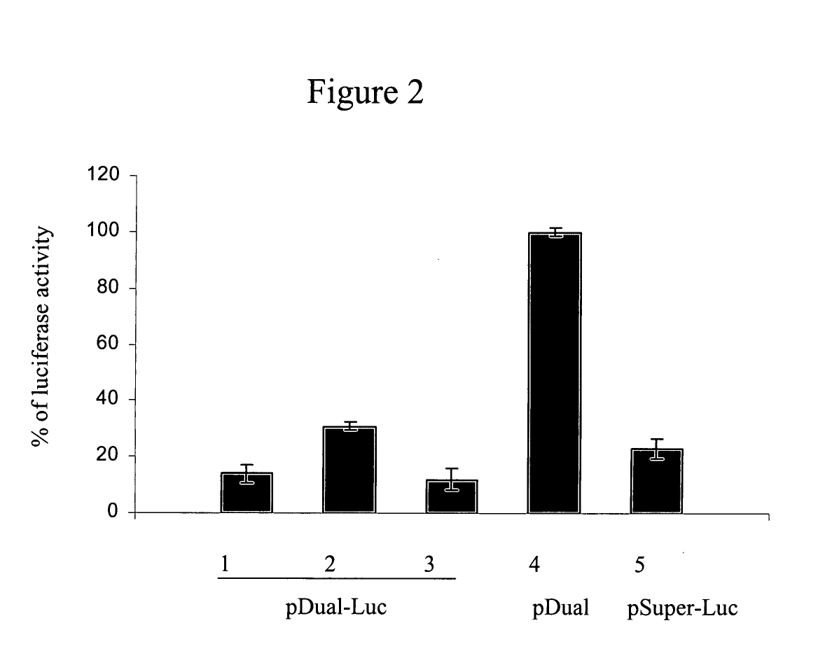

Efficient inhibition of both transfected and endogenous gene expression. To determine whether the pDual system can efficiently produce functional siRNAs, a reporter gene assay using firefly luciferase was used. siRNA sequences 19 nt in length that correspond to three different regions of the firefly luciferase gene were chosen as the target sequences and cloned into pDual. For comparison, one of the target sequences was also used to prepare a short hairpin siRNA using the pSUPER vector. These siRNA expression vectors were cotransfected with firefly luciferase and Renilla luciferase expression vectors into 293T cells at a ratio of 20:2:1. Cells were lysed 48 hours after transfection and firefly luciferase and Renilla luciferase activity were measured with the Dual-Luciferase Reporter assay system (Promega). As shown in FIG. 2, siRNAs transcribed by pDual system that corresponding to sequences 1, 2, 3 (see Methods below) can efficiently suppress transfected luciferase gene activity with a reduction of 80 to 90% in gene expression compared to the empty pDual control (4). These results are similar to those resulting from the hairpin siRNA expressed by the single H1 promoter construct (5). Renilla luciferase activity was not affected by the expression of these siRNAs targeting firefly luciferase coding sequences, showing that the effect is gene specific.

We next determined whether siRNAs expressed by the pDual system can efficiently inhibit endogenous gene activity. We first examined suppression of expression of the nuclear protein lamin A/C. It has been shown that lamin A/C expression can be efficiently suppressed by synthetic siRNAs or hairpin siRNAs transcribed by a single U6 promoter. See, e.g., Elbashir, S. M., et al. (2001) Nature 411, 494-8; Paul, C. P., et al. (2002) Nat Biotechnol 20, 505-8. A pair of oligonucleotides that contain 21nt siRNA coding sequences for lamin A/C were inserted into pDual to generate the pDual-lamin A/C. HeLa cells were then transfected with either pDual-lamin A/C or the empty pDual vector as a control. In order to identify the transfected cells, cells were also cotransfected with pCMV-GFP. Lamin A/C expression level was significantly reduced in cells transfected with pDual-lamin A/C (indicated by GFP). In some cells, lamin A/C expression was completely abolished. This is in striking contrast to cells transfected with pDual alone, where no detectable change in lamin A/C expression was found.

Suppression of a second endogenous gene, neuron specific βIII-tubulin, which is expressed in differentiated neuronal cells, was also examined. Pluripotent P19 mouse embryonic carcinoma cells can be induced to differentiate into neurons by ectopic expression of MASH1, a bHLH transcription factor. βIII-tubulin is an abundant and readily detectable protein marker in differentiated P19 neurons. A pair of oligonucleotides that contain 19 bp siRNA coding sequences for βIII-tubulin were inserted into pDual vector to generate the pDual-βIIIT. P19 cells were cotransfected with either pDual-βIIIT and biCS2-MASH1/GFP, or pDual and biCS2-MASH1/GFP. The cells were fixed and immunostained with TuJ1 antibody (against βIII-tubulin) five days after transfection. Ectopic expression of MASH1 in P19 cells induced neuronal differentiation and the expression of βIII-tubulin in differentiated neuronal cells, while the co-expression of siRNA from pDual-βIIIT can efficiently reduce the number of neuronal cells that express βIII-tubulin (the GFP expressions in both cases were similar. These results demonstrate that siRNAs transcribed in vivo by two opposing RNA pol III promoters in the pDual system can specifically and efficiently suppress endogenous gene expression.

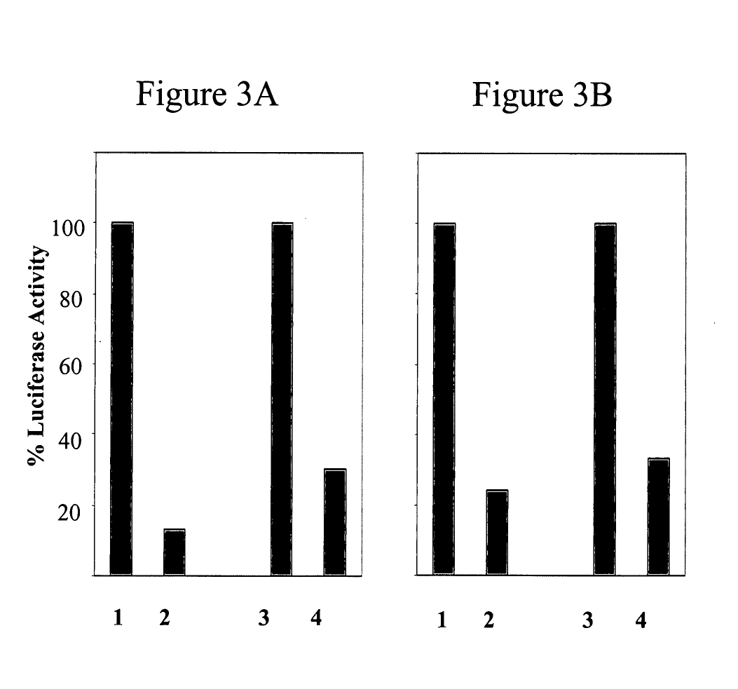

Gene suppression activity of the PCR-derived siRNA expression cassettes. To determine the suppression efficiency of the PCR-derived siRNA expression cassettes derived from the pDual system, they were tested with the transfected luciferase gene and the results were compared with the siRNA expression vector system in two different cell lines. Either the PCR product or pDual-luc vector was contransfected into HEK-293T or P19 cells with pGL3 and pRL-SV40 in a ratio of 10:1:0.5. As shown in FIGS. 3A and 3B, the PCR product inhibits the expression of the transiently transfected luciferase gene by about 70-80% (4 in FIGS. 3A and B), in comparison to 80-90% suppression in cells transfected with pDual-luc (2 in FIGS. 3A and B). In these experiments, the same amounts of PCR product and pDual-luc were used. Even though the molar ratio of PCR product to the reporter gene is much higher than the ratio of pDual-luc to the reporter, the suppression efficiency of the PCR product is still slightly lower than that of the vector-based approach. This could result from lower transfection efficiency, lower transcriptional efficiency with the PCR product and/or its instability in the cells.

We next determined whether the PCR product could suppress endogenous gene activity. The PCR-derived lamin A/C siRNA expression cassettes were again cotransfected with pCMV-GFP into the HeLa cells and the luciferase siRNA expression cassette was used as a control. Three days after transfection, cells were fixed and immunostained with anti-lamin A/C antibody. Cells transfected with lamin A/C siRNA expression cassettes have significantly reduced lamin A/C expression, while the control luciferase siRNA expression cassette had no effect on lamin A/C expression. These results demonstrate that the PCR-derived siRNA cassettes based on our pDual system are sufficiently stable in cells to temporally suppress endogenous gene expression.

The dual promoter system can be used to express long double strand RNAs in cells. In mammalian somatic cells, long double-stranded RNA can activate the dsRNA dependent kinase PKR, which phosphorylates EIF2α, resulting in nonspecific shut down of translation (Gil, J. & Esteban, M. (2000) Apoptosis 5, 107-14; Baglioni, C. & Nilsen, T. W. (1983) Journal of Biological Chemistry 258, 2118-21; D'Alessandro, et al. (1983) Journal of Interferon Research 3, 465-71). dsRNA also activates 2′-5′ oligoadenylate polymerase, the product of which is an essential cofactor for the nonspecific ribonuclease, RNase L. See, e.g., Baglioni, C. (1986) Journal of Biological Chemistry 261, 338-42. Because dsRNAs longer than 30 bp cause nonspecific cytotoxic effects in mammalian cells by these mechanisms, long dsRNAs have limited utility as initiators of RNA interference in mammalian somatic cells. Nonetheless, it has been shown that in P19 cells, dsRNAs over 500 bp in length can trigger gene specific RNA interference without nonspecific cytotoxic effects. See, e.g., Paddison, P. J., Caudy, A. A. & Hannon, G. J. (2002) Proc Natl Acad Sci U S A 99, 1443-8. It has also been shown that, in differentiated neuronal or muscle cell lines (Gan, L., et al. (2002) J Neurosci Methods 121, 151-7; Yi, et al. (2003) J Biol Chem 278, 934-9), longer dsRNA can be used to inhibit gene function.

In order to test whether the dual promoter vector can be used to express long dsRNAs, a series of pDual vectors that contain 56, 120, 218, 300 bp fragments of the luciferase coding region were constructed (these sequences do not contain four or more consecutive As or Ts). Fragments of the luciferase coding region were first amplified by PCR reactions, and 20 nt and 22 nt flanking sequences, identical to those used in the above PCR-derived siRNA expression cassettes, were added in the primers used for amplification. To insert these sequences into the pDual vector, the PCR fragments were digested with Hind III and BglII and ligated into pDual digested with the same enzymes. To produce long double strand RNA expression cassettes, these PCR fragments were used to bridging the H1 and U6 promoters. PCR reactions were performed in a similar manner using the two annealing temperature PCR protocol. More than 95% the final PCR products were the full length siRNA expression cassettes.

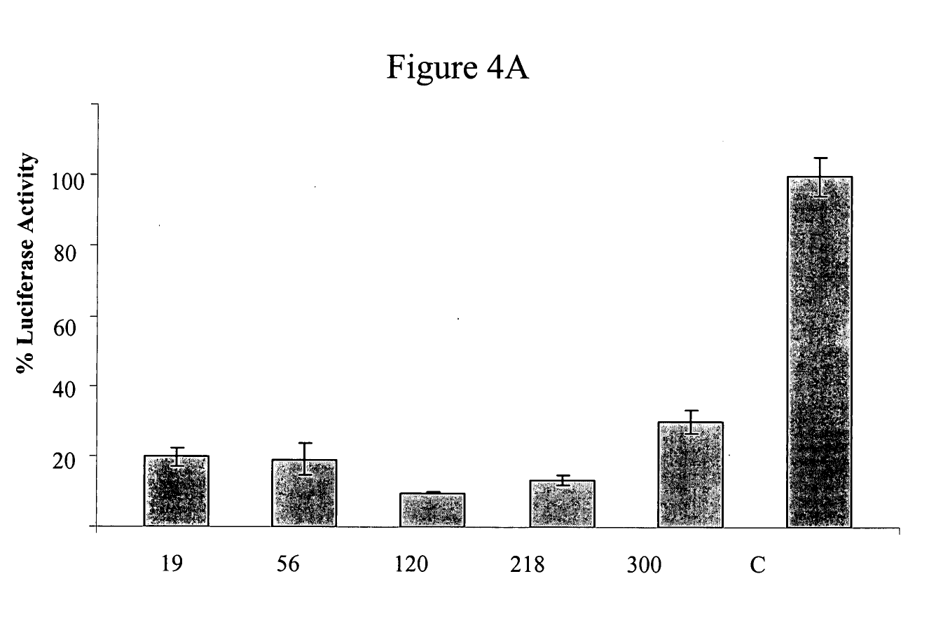

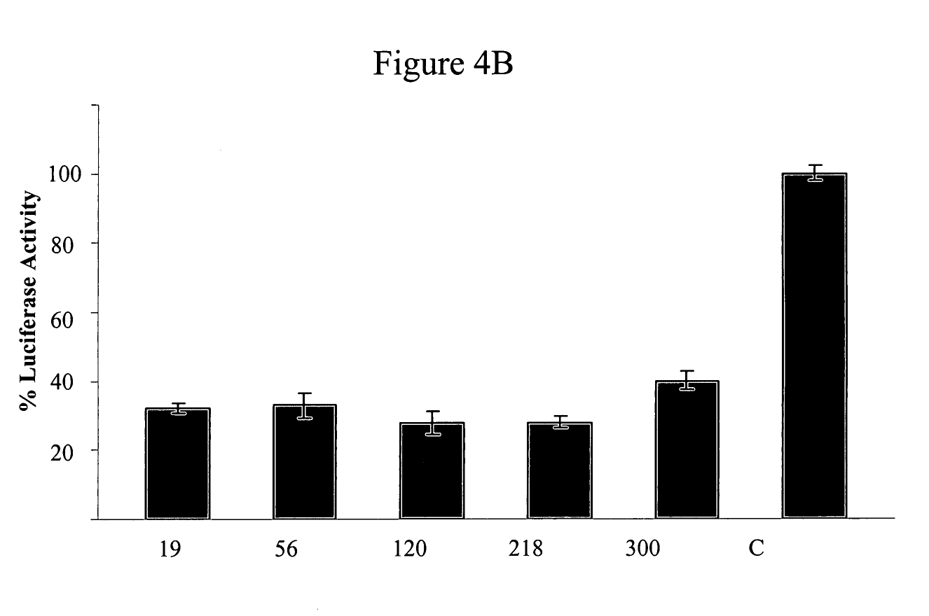

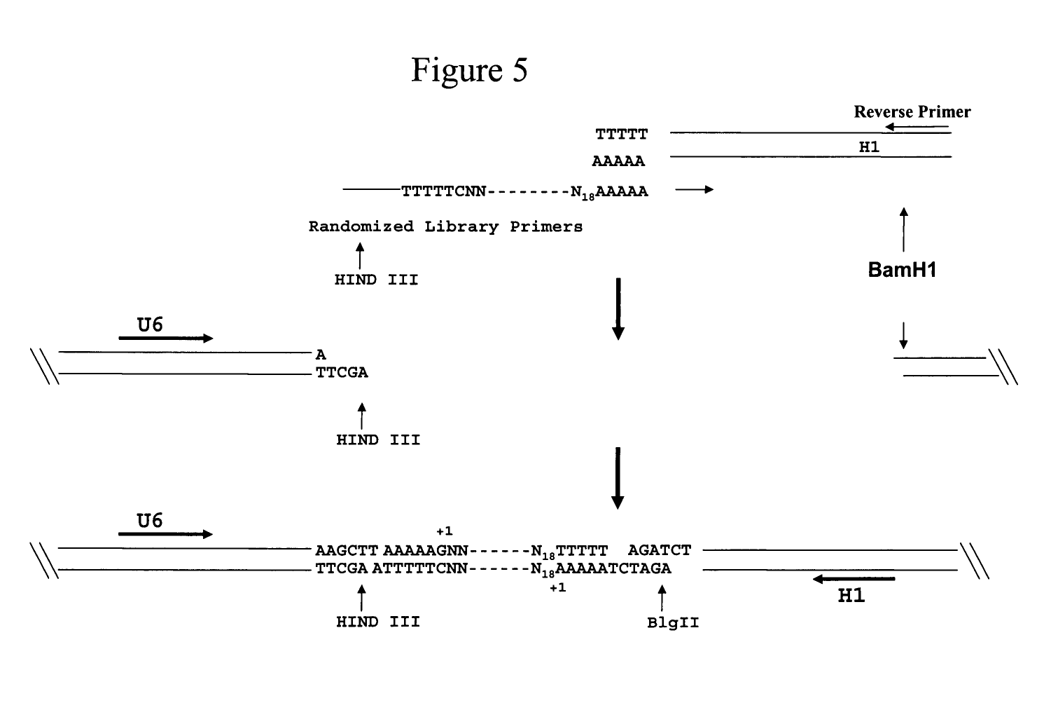

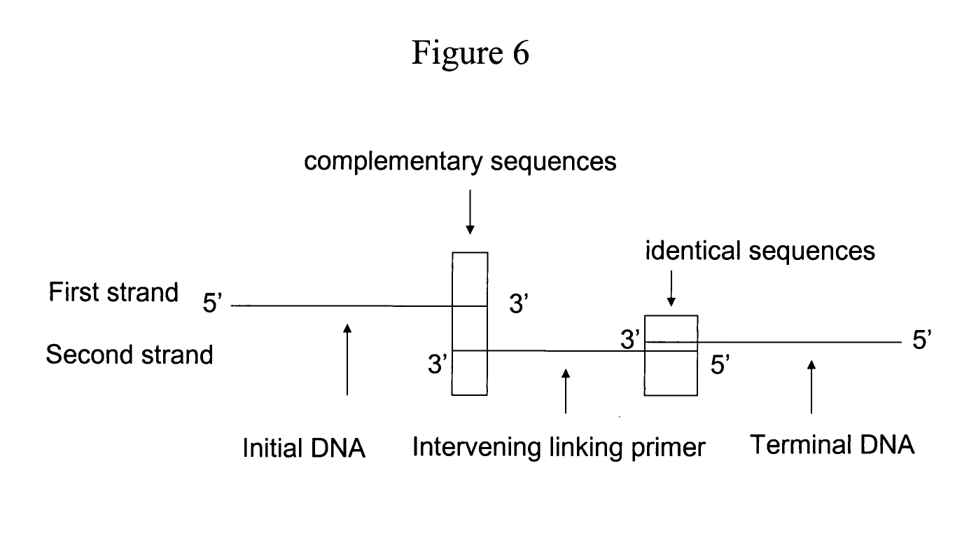

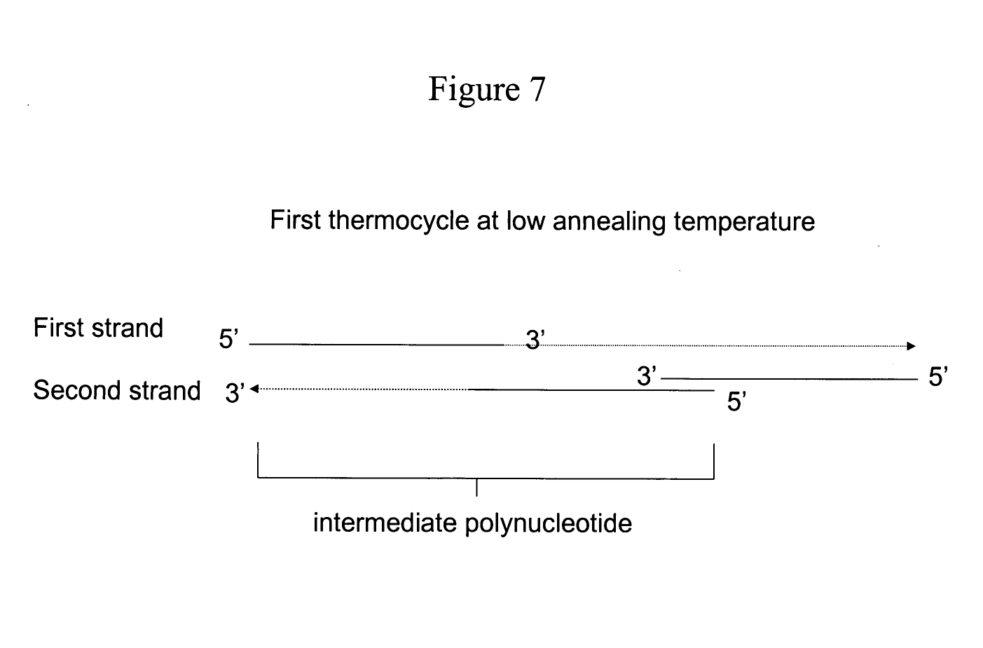

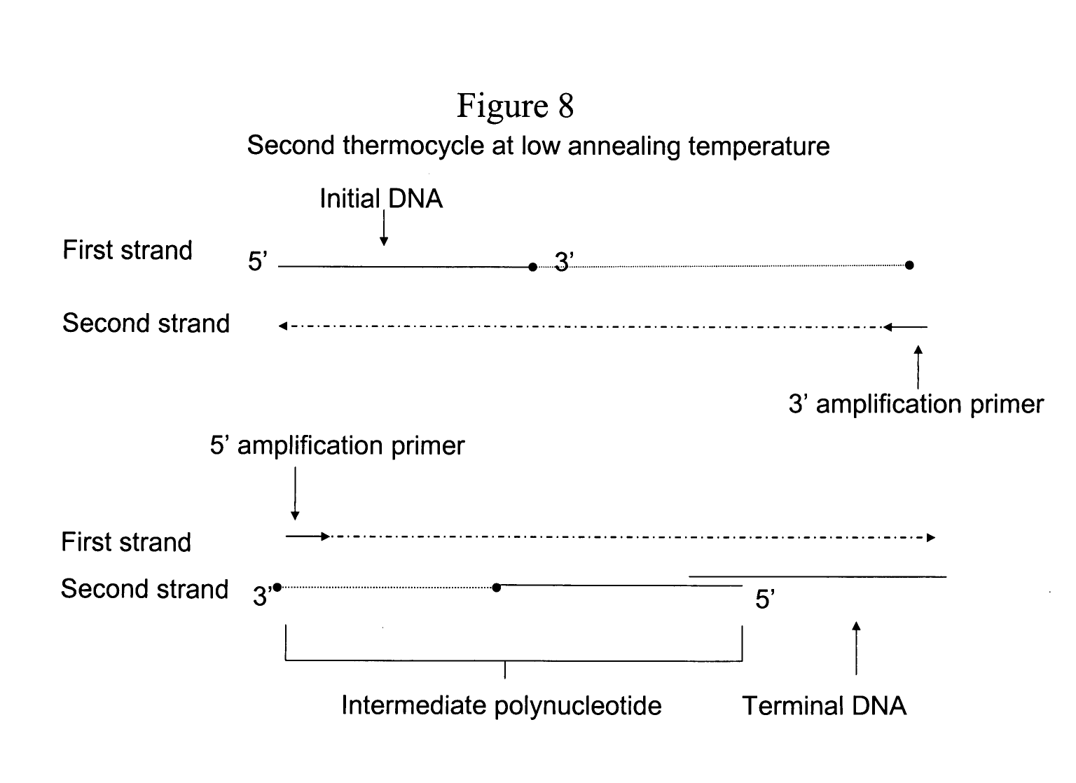

To test the gene suppression activity of these long siRNA expression vectors or the PCR-derived siRNA expression cassettes, they were cotransfected with reporter plasmids pGL3-luc and pRL-SV40 into murine P19 embryonal carcinoma cell. As shown in FIGS. 4A and 4B, these long siRNA expressed in vivo using either pDual or PCR fragments can significantly and specifically inhibit luciferase expression. These results are similar to those obtained from the short siRNA expressed by the pDual or PCR fragment that contains only the 19 bp luciferase coding sequence. However, when these fragments were transfected into 293T cells, cytotoxic effects were observed, especially for the double-stranded siRNA longer than 50 bp. These results suggest that both the H1 and U6 promoters in the pDual vector are functional efficiently, and can produce enough double strand RNA molecules to trigger cytotoxic effects.

We also generated a library of siRNA expression cassettes in 384 microtiter plates. Each well of the plates contained a different third primer designed to suppress expression from a different gene. The expression cassettes were generated using the PCR methods described in this example. This siRNA expression cassette library is useful for genome-wide screening as an alternative to non-specific mutagenesis methods.

We have designed and constructed a novel dual promoter system (pDual) for expression of siRNAs in mammalian cells which can specifically and efficiently suppress gene functions. Compared to previously reported single-promoter, hairpin-RNA based systems, the pDual system offers several advantages: (1) for cloning of siRNA expression vector, shorter insert encoding gene specific sequences can be used (lower cost); (2) because the inserted sequences does not have hairpin structure, cloning and sequencing processes are much easier (higher successful rate); (3) pDual system allows building a siRNA library, in which the gene specific sequences between the two pol III promoters can be randomized or derived from mRNA. Such a library (the counterpart of cDNA library) may be useful in screening all genes that are involved in any specific biological pathway.

Based on this vector, we have developed a single step PCR protocol to produce siRNA expression cassettes in a high throughput fashion. We have shown that these siRNA expression cassettes can also specifically and efficiently suppress the expression of both transfected and endogenous genes. Compared to all available approaches reported previously, our single-step PCR approach using one gene-specific oligonucleotide is highly cost effective, and most importantly it makes high throughput production of siRNA expression system feasible and allows genome-wide functional gene annotation by gene knockdown approach. The PCR fragments can also be easily transferred into vector based library using restriction digestion and ligation, or recombination.

Materials and Methods

Construction of plasmids for siRNA synthesis. To create the dual promoter vector (pDual) for siRNA expression, the promoter region of mouse small nuclear RNA U6 and the promoter region of human H1 RNA (the RNA component of RNase PI), were amplified by PCR and cloned into pBluescript SK vector in opposite directions as shown in FIG. 1A. A Hind III site upstream of the U6 RNA transcription start site, and a BglII site upstream of the H1 RNA transcription start site, were created by site-directed mutagenesis (Stratgene). To construct the gene specific siRNA expression plasmids, a pair of 35 to 37 base oligonucleotides were annealed and ligated into pDual digested with Bgl II and Hind III (FIG. 1A). These oligonucleotides contain 19 to 21 nt gene specific sequences flanked by five As on the 5′ side and five Ts on the 3′ side, as well as the restriction sites (Bgl II and Hind III).

The sequences for the siRNA encoding oligonucleotides are as follows:

(1) (firefly luciferase)
sense oligo-
agcttaaaaagacgaacacttcttcatcgttttta,
antisense oligo-
gatctaaaaacgatgaagaagtgttcgtcttttta;
(2) (firefly luciferase)
sense oligo-
agcttaaaaagttcgt-cacatctcatctacttttta,
antisense oligo-
gatctaaaaagtagatgagatgtgacgaacttttta;
(3) (firefly luciferase)
sense oligo-
agcttaaaaagtgcgctgctggtgccaacttttta,
antisense oligo-
gatctaaaaa-gttggcaccagcagcgcacttttta;
(4) (lamin A/C)
sense oligo-
agcttaaaaagatgttcttctggaagtcca-gttttta,
antisense oligo-
gatctaaaaactggacttccagaagaacatcttttta;
(5) (Tuj 1)
sense oligo-
a-gcttaaaaagagtccacttggctctgtcttttta,
antisense oligo-
gatctaaaaagacagagccaagtggactcttttta.

For expression of hairpin siRNA for firefly luciferase, the following two oligonucleotides are annealed and cloned into pSuper (Oligoengine) (Brummelkamp, T. R., et al. (2002) Science 296, 550-3):

gatccccgacgaacacttcttcatcgttcaagagacgatgaagaagtgttcgtctttttggaaa,
agcttttccaaaaagacgaacacttcttcatcgtctcttgaacgatgaagaagtgttcgtcggg.

Transfection and gene silencing reporter assays. P19 mouse embryonic carcinoma cells (American Type Tissue Culture Collection, CRL-1825) were cultured in MEM-α (GIBCO/BRL) supplemented with 10% fetal bovine serum. HEK-293T and Hela cells were cultured in DMEM supplemented with 10% fetal bovine serum. P19 and HEK-293T cells were transfected with Fugene 6 (Roche Biochemicals); HeLa cells were transfected with lipofectamine 2000 (Invitrogen), as directed by the manufacturers. For gene silencing experiments with transfected firefly luciferase, pGL3 and pRL-SV40 were cotransfected with the siRNA expression plasmids or PCR fragments at a ratio of 2:1:20. Cells were lysed 48 hours after transfection. Luciferase activities were measured with the Dual-Luciferase Reporter assay system (Promega). Renilla luciferase activity was used as a control for normalization.

Immunohistochemistry. Cells were cultured and transfected in 96 well plates, and 3 to 5 days after transfection the cells were fixed with 4% paraformaldehyde in phosphate buffered saline (PBS) for 20 minutes at room temperature. The cells were washed with PBS three times, and then stained overnight at 4° C. with primary antibody diluted with PBS containing 0.3% Triton X-100 and 5% horse serum. Monoclonal mouse anti-lamin A/C antibody (Santa Cruz Biotech) was used at dilution of 1:300. Monoclonal mouse antibody against neuronal class III β-tubulin (Covance Inc.) was used at dilution of 1:500. After washing with PBS containing 0.1% Triton X-100, cells were incubated for two hours with the secondary antibody, Cy3-Donkey anti-mouse IgG (Jackson Immuno Research). Cells were then washed three times with PBS. Images were analyzed by fluorescence microscopy (Nikon eclipse TE2000-U) and photographed with a digital camera.

Polymerase chain reaction for production of siRNA expression cassettes. The U6 promoter and H1 promoter sequences were preamplified by PCR from siRNA expression plasmid for luciferase using the following primers: H1 forward-gtaatacgaCtcactatgcgaacgctgacgtcatcaac; H1 reverse-tttttagatctgtctcatacag; U6 forward-GGAATCAGCTATGACCATgTTACgATCCGACGCCGCCATCTC; U6 reverse-ctttttaagcttttctccaagg. To produce siRNA expression cassettes (FIG. 1B), the PCR reactions were performed with H1 and U6 promoter as templates, two universal primers and one gene specific primer. The two universal primers are common for all PCR reactions: the universal forward primer is complementary to the 5′ end of the U6 promoter (GGAATCAGCTATGACCATgTTACgATCCG), and the universal reverse primer is complementary to the 5′ end of the H1 promoter (gtaatacgaCtcactatgcgaacgctgacG). The gene specific primer is unique for each siRNA expression cassette: the 5′ region of this oligonucleotide contains a 22 nt sequence complementary to the H1 promoter sequence (ctgtatgagacagatctaaaaa). This sequence is followed by a 19 nt siRNA encoding sequence, and then a 20 nt sequence complementary to the U6 promoter sequence (tttttaagcttttctccaag). All the PCR reactions were carried out as follows: 3 min at 94° C.; 1 min at 94° C., 1 min at 55° C., and 1 min at 72° C. for 5 cycles; followed by 1 min at 94° C., 1 min at 63° C., and 1 min at 72° C. for 36 cycles. The resulting PCR products are approximately 650 base pairs and with over 95% purity as judged by agarose gel electrophoresis. The PCR products were then purified with a Qiaquick PCR purification kit (Qiagen).

The above example is provided to illustrate the invention but not to limit its scope. Other variants of the invention will be readily apparent to one of ordinary skill in the art and are encompassed by the appended claims. All publications, databases, Genbank sequences, patents, and patent applications cited herein are hereby incorporated by reference.

1. A method for producing two complementary strands of a tripartite DNA comprising a 5′ amplification primer binding site and a 3′ amplification primer binding site with an intervening sequence between the two sites, the method comprising,
providing a polymerase extension reaction mixture comprising the following reagents:
a) an initial double-stranded DNA comprising
a left end comprising a 5′ amplification primer binding site; and
a right end complementary to a 3′ end of an intervening linking primer;
b) the intervening linking primer comprising:
a 5′ end complementary to a left end of a terminal DNA;
a 3′ end complementary to the right end of the initial DNA; and
an intervening sequence between the 5′ end and the 3′ end;
c) the terminal double-stranded DNA comprising
a right end that is a 3′ amplification primer binding site; and
a left end complementary to the 5′ end of the intervening linking primer;
d) DNA polymerase and components sufficient for a polymerase chain reaction;
reacting the reagents of the mixture in at least two thermocycles, wherein
the first cycle comprises
a melting temperature to denature double-stranded DNA;
an initial annealing temperature to anneal the intervening linking primer to a strand of the initial double-stranded DNA; and
an extension temperature for the polymerase to extend a 3′ end of the strand of the initial double-stranded DNA using the intervening linking primer as a template to produce an intermediate product comprising the strand of the initial double stranded DNA and a complement of the intervening linking primer; and
the second cycle comprises
a melting temperature to denature double-stranded DNA;
an annealing temperature to anneal the intermediate product to a strand of the terminal double-stranded DNA; and
an extension temperature for the polymerase
to extend the 3′ end of the intermediate product using the strand of the terminal double-stranded DNA as a template to produce a single stranded tripartite DNA comprising the strand of the initial double stranded DNA, the complement of the intervening linking primer, and the complement of the strand of the terminal double stranded DNA sequences; and
to extend the 3′ end of the strand of the terminal double stranded DNA using the intermediate product as a template to produce the complement of the single-stranded tripartite DNA.

2. The method of claim 1, wherein the polymerase extension reaction mixture further comprises
a) a 5′ amplification primer that anneals to the 5′ amplification primer binding site; and
b) a 3′ amplification primer that anneals to the 3′ amplification primer binding site; and
and the method further comprises at least one additional thermocycle following the second thermocycle, wherein the additional thermocycle comprises a differential annealing temperature at which the 5′ amplification primer anneals to the 5′ amplification primer binding site and the 3′ amplification primer anneals to the 3′ amplification primer binding site, but the intervening linking primer does not anneal to the strand of the initial double-stranded DNA, thereby amplifying the two complementary strands of a tripartite DNA.

3. The method of claim 1, wherein the initial DNA comprises a first pol III promoter and the terminal DNA comprises a second pol III promoter; and
the method links the initial and terminal DNAs such that the promoters are oriented towards each other.

4. The method of claim 4, wherein the initial DNA and terminal DNA each further comprise a pol III terminator.

5. The method of claim 3, wherein the first pol III promoter is the U6 promoter and the second pol III promoter is the H1 promoter.

6. The method of claim 1, further comprising cloning the linked DNA sequences into a vector.

7. The method of claim 1, wherein the 5′ and 3′ amplification primers comprise restriction enzyme recognition sequences.

8. The method of claim 7, comprising cleaving the tripartite DNA sequences with a restriction enzyme that cleaves at least one restriction enzyme recognition sequence and ligating the cleaved DNA sequence into a vector.

9. The method of claim 1, wherein the mixture comprises a plurality of different intervening linking primers comprising different intervening sequences.

10. The method of claim 9, wherein the different intervening linking primers comprise randomly-generated intervening sequences.

11. The method of claim 10, wherein the initial DNA comprises a first pol III promoter and pol III terminator and the terminal DNA comprises a second pol III promoter and pol III terminator;
the method links the polynucleotide sequences such that the promoters are oriented towards each other; and
the resulting tripartite DNAs are cloned into a vector, thereby synthesizing a library of random intervening sequences between the first and second pol III promoters.

12. The method of claim 1, further comprising introducing the amplified sequences into cells under conditions resulting in expression from the pol III promoters in the cell, thereby forming double-stranded RNA.

13. The method of claim 1, wherein the intervening linking primer is between 40-70 nucleotides long.

14. The method of claim 13, wherein the intervening linking primer is between 61-63 nucleotides long.

15. The method of claim 1, wherein the initial annealing temperature is about 55° C.

16. The method of claim 1, wherein the differential annealing temperature is about 63° C.

17. The method of claim 1, wherein the initial DNA and the terminal DNA are part of one polynucleotide.

18. The method of claim 1, wherein the initial DNA and the terminal DNA are part of different polynucleotides.

19. A kit for generating a siRNA polynucleotide, the kit comprising,
a) an initial double-stranded DNA comprising
a left end comprising a 5′ amplification primer binding site; and
a right end complementary to a 3′ end of an intervening linking primer;
b) the intervening linking primer comprising:
a 5′ end complementary to a left end of a terminal DNA; and
a 3′ end complementary to the right end of the initial DNA;
an intervening sequence between the 5′ end and the 3′ end;
c) the terminal double-stranded DNA comprising
a right end that is a 3′ amplification primer binding site; and
a left end complementary to the 5′ end of the intervening linking primer;
d) a 5′ amplification primer; and
e) a 3′ amplification primer.

20. The kit of claim 19, wherein the initial DNA comprises a first pol III promoter and the terminal DNA comprises a second pol III promoter.

21. The kit of claim 23, wherein the initial DNA and terminal DNA each further comprise a pol III terminator.

22. The kit of claim 19, wherein the kit further comprises a DNA polymerase.

23. The kit of claim 19, wherein the 5′ and 3′ amplification primers comprise restriction enzyme recognition sequences.

24. The kit of claim 19, wherein the kit comprises a plurality of different intervening linking primers comprising different intervening sequences.

25. The kit of claim 24, wherein the different intervening linking primers comprise randomly-generated intervening sequences.

26. The kit of claim 20, wherein the first pol III promoter is the U6 promoter and the second pol III promoter is the H1 promoter.

27. The kit of claim 19, wherein the intervening linking primer is between 40-70 nucleotides long.

28. The kit of claim 27, wherein the intervening linking primer is between 61-63 nucleotides long.

29. The kit of claim 19, wherein the initial DNA and the terminal DNA are part of one polynucleotide.

30. The kit of claim 19, wherein the initial DNA and the terminal DNA are part of different polynucleotides.

31. A mixture comprising,
a) an initial double-stranded DNA comprising
a left end comprising a 5′ amplification primer binding site; and
a right end complementary to a 3′ end of an intervening linking primer;
b) the intervening linking primer comprising:
a 5′ end complementary to a left end of a terminal DNA; and
a 3′ end complementary to the right end of the initial DNA;
an intervening sequence between the 5′ end and the 3′ end;
c) the terminal double-stranded DNA comprising
a right end that is a 3′ amplification primer binding site; and
a left end complementary to the 5′ end of the intervening linking primer;
d) a 5′ amplification primer; and
e) a 3′ amplification primer.

32. The mixture of claim 31, wherein the initial DNA comprises a first pol III promoter and the terminal DNA comprises a second pol III promoter.

33. The mixture of claim 32, wherein the initial DNA and terminal DNA each further comprise a pol III terminator.

34. The mixture of claim 31, wherein the kit further comprises a DNA polymerase.

35. The mixture of claim 31, wherein the 5′ and 3′ amplification primers comprise restriction enzyme recognition sequences.

36. The mixture of claim 31, wherein the mixture comprises a plurality of different intervening linking primers comprising different intervening sequences.

37. The mixture of claim 36, wherein the different intervening linking primers comprise randomly-generated intervening sequences.

38. The mixture of claim 32, wherein the first pol III promoter is the U6 promoter and the second pol III promoter is the H1 promoter.

39. The mixture of claim 31, wherein the intervening linking primer is between 40-70 nucleotides long.

40. The mixture of claim 39, wherein the intervening linking primer is between 61-63 nucleotides long.

41. The mixture of claim 31, wherein the initial and terminal DNAs are part of one polynucleotide.

42. The mixture of claim 31, wherein the initial and terminal DNAs are part of different polynucleotides.


SEQUENCE LISTING (28 sequences)

SEQ ID NO: 1 - DNA, 14 bp, Artificial Sequence
  Description of Artificial Sequence5′ portion of annealed and ligated complementary oligonucleotide PCR product
aagcttaaaaagnn

SEQ ID NO: 2 - DNA, 29 bp, Artificial Sequence
  Description of Artificial Sequence3′ portion of annealed and ligated complementary oligonucleotide PCR product
nnnnnnnnnnnnnnnnnntttttagatct

SEQ ID NO: 3 - DNA, 13 bp, Artificial Sequence
  Description of Artificial Sequence5′ portion of gene specific oligo
agcttaaaaagnn

SEQ ID NO: 4 - DNA, 24 bp, Artificial Sequence
  Description of Artificial Sequence3′ portion of gene specific oligo
nnnnnnnnnnnnnnnnnnttttta

SEQ ID NO: 5 - DNA, 28 bp, Artificial Sequence
  Description of Artificial Sequence5′ portion of gene specific oligo
gatctaaaaannnnnnnnnnnnnnnnnn

SEQ ID NO: 6 - DNA, 17 bp, Artificial Sequence
  Description of Artificial Sequence5′ siRNA portion and a few complementary nucleotides of “third” oligonucleotide primer, gene specific primer-X, or intervening linking primer
aaaaannnnnnnnnnnn

SEQ ID NO: 7 - DNA, 23 bp, Artificial Sequence
  Description of Artificial Sequence3′ siRNA portion and a few complementary nucleotides of “third” oligonucleotide primer bridging U6 and H1 promoter fragments in siRNA expression cassette
nnnnnnnnnnnnnnnnnnttttt

SEQ ID NO: 8 - DNA, 23 bp, Artificial Sequence
  Description of Artificial Sequence3′ portion of chemically synthesized third primer with randomized siRNA sequence, randomized library primer
nnnnnnnnnnnnnnnnnnaaaaa

SEQ ID NO: 9 - DNA, 35 bp, Artificial Sequence
  Description of Artificial Sequencefirefly luciferase siRNA encoding oligonucleotide (1) sense oligo
agcttaaaaagacgaacacttcttcatcgttttta

SEQ ID NO: 10 - DNA, 35 bp, Artificial Sequence
  Description of Artificial Sequencefirefly luciferase siRNA encoding oligonucleotide (1) antisense oligo
gatctaaaaacgatgaagaagtgttcgtcttttta

SEQ ID NO: 11 - DNA, 36 bp, Artificial Sequence
  Description of Artificial Sequencefirefly luciferase siRNA encoding oligonucleotide (2) sense oligo
agcttaaaaagttcgtcacatctcatctacttttta

SEQ ID NO: 12 - DNA, 36 bp, Artificial Sequence
  Description of Artificial Sequencefirefly luciferase siRNA encoding oligonucleotide (2) antisense oligo
gatctaaaaagtagatgagatgtgacgaacttttta

SEQ ID NO: 13 - DNA, 35 bp, Artificial Sequence
  Description of Artificial Sequencefirefly luciferase siRNA encoding oligonucleotide (3) sense oligo
agcttaaaaagtgcgctgctggtgccaacttttta

SEQ ID NO: 14 - DNA, 35 bp, Artificial Sequence
  Description of Artificial Sequencefirefly luciferase siRNA encoding oligonucleotide (3) antisense oligo
gatctaaaaagttggcaccagcagcgcacttttta

SEQ ID NO: 15 - DNA, 37 bp, Artificial Sequence
  Description of Artificial Sequencelamin A/C siRNA encoding oligonucleotide (4) sense oligo
agcttaaaaagatgttcttctggaagtccagttttta

SEQ ID NO: 16 - DNA, 37 bp, Artificial Sequence
  Description of Artificial Sequencelamin A/C siRNA encoding oligonucleotide (4) antisense oligo
gatctaaaaactggacttccagaagaacatcttttta

SEQ ID NO: 17 - DNA, 35 bp, Artificial Sequence
  Description of Artificial SequenceTuj1 siRNA encoding oligonucleotide (5) sense oligo
agcttaaaaagagtccacttggctctgtcttttta

SEQ ID NO: 18 - DNA, 35 bp, Artificial Sequence
  Description of Artificial SequenceTuj1 siRNA encoding oligonucleotide (5) antisense oligo
gatctaaaaagacagagccaagtggactcttttta

SEQ ID NO: 19 - DNA, 64 bp, Artificial Sequence
  Description of Artificial Sequencefirefly luciferase hairpin siRNA oligonucleotide annealed and cloned into pSuper
gatccccgacgaacacttcttcatcgttcaagagacgatgaagaagtgttcgtctttttggaaa

SEQ ID NO: 20 - DNA, 64 bp, Artificial Sequence
  Description of Artificial Sequencefirefly luciferase hairpin siRNA oligonucleotide annealed and cloned into pSuper
agcttttccaaaaagacgaacacttcttcatcgtctcttgaacgatgaagaagtgttcgtcggg

SEQ ID NO: 21 - DNA, 38 bp, Artificial Sequence
  Description of Artificial SequencePCR preamplification primer H1 forward
gtaatacgactcactatgcgaacgctgacgtcatcaac

SEQ ID NO: 22 - DNA, 22 bp, Artificial Sequence
  Description of Artificial SequencePCR preamplification primer H1 reverse
tttttagatctgtctcatacag

SEQ ID NO: 23 - DNA, 42 bp, Artificial Sequence
  Description of Artificial SequencePCR preamplification primer U6 forward
ggaatcagctatgaccatgttacgatccgacgccgccatctc

SEQ ID NO: 24 - DNA, 22 bp, Artificial Sequence
  Description of Artificial SequencePCR preamplification primer U6 reverse
ctttttaagcttttctccaagg

SEQ ID NO: 25 - DNA, 29 bp, Artificial Sequence
  Description of Artificial Sequenceuniversal forward primer complementary to 5′ end of U6 promoter
ggaatcagctatgaccatgttacgatccg

SEQ ID NO: 26 - DNA, 30 bp, Artificial Sequence
  Description of Artificial Sequenceuniversal reverse primer complementary to 5′ end of H1 promoter
gtaatacgactcactatgcgaacgctgacg

SEQ ID NO: 27 - DNA, 22 bp, Artificial Sequence
  Description of Artificial Sequencegene specific primer, 5′ region complementary to H1 promoter
ctgtatgagacagatctaaaaa

SEQ ID NO: 28 - DNA, 20 bp, Artificial Sequence
  Description of Artificial SequencesiRNA encoding sequence and sequence complementary to U6 promoter
tttttaagcttttctccaag